(19) United States
(12) Patent Application Publication
(10) Pub. No.: US 2020/0194126 A1
(43) Pub. Date: Jun. 18, 2020

(54) SYSTEMS AND METHODS FOR PROFILING AND CLASSIFYING HEALTH-RELATED FEATURES

(71) Applicant: The Regents of the University of California, Oakland, CA (US)

(72) Inventors: Ryan Lim, Irvine, CA (US); Sarah Hernandez, San Juan Capistrano, CA (US)

(73) Assignee: The Regents of the University of California, Oakland, CA (US)

(21) Appl. No.: 16/711,945

(22) Filed: Dec. 12, 2019

Related U.S. Application Data
(60) Provisional application No. 62/780,528, filed on Dec. 17, 2018.

(51) Int. Cl. G16H 50/30; G16H 10/60; G16H 20/60; G16H 20/30; G16H 20/90; C12Q 1/6869

(52) U.S. Cl. CPC G16H 50/30; G16H 10/60; C12Q 1/6869; G16H 20/30; G16H 20/90; G16H 20/60

(57) ABSTRACT

Embodiments of the present systems and methods may provide techniques that may profile and quantify the microbiome and metabolome and identify the novel health, lifestyle, and environmental-related proteins that they affect. Embodiments may provide the capability for the classification of patients or other biological entities into clinical or non-clinical but related groups and labels, based on assessment of their microbiome and metabolome. Embodiments may provide the capability to assess patient health, identify disease risk factors, identify, and rank therapeutic targets, determine the functional contributions of the microbiome to patient health, and even predict outcomes such as disease development and drug response. Other embodiments may provide consumers with lifestyle related information and comparisons with other consumers' data, potentially allowing consumers to tailor lifestyle choices such as nutrition, exercise, and supplementation. Furthermore, other embodiments may provide health assessments that pertain to animal or environmental related entities.

### CROSS-REFERENCE TO RELATED APPLICATIONS

This application claims the benefit of U.S. Provisional Application No. 62/780,528, filed Dec. 17, 2018, the contents of which are incorporated herein in their entirety.

### TECHNICAL FIELD OF THE INVENTION

The present invention relates to techniques for profiling and quantifying the microbiome and metabolome and identifying the novel health-related proteins that they affect.

### BACKGROUND OF THE INVENTION

Only 43% of cells and 1% of DNA within the human body are of human origin. The remaining contribution comes from bacterial, viral, and fungal species, the collection of which is called the microbiome. In addition to the microbiome, a wealth of information is also contained in the metabolome, or the collection of small molecules, partially generated by the microbiome. The metabolome is largely comprised of naturally-produced metabolites, though it also may include short peptides and oligonucleotides produced by the residents of the microbiome.

Due to the biological functions of these microbes within the human body, both the microbiome and metabolome play an important role in disease pathogenesis, health outcomes, and drug response. Despite this, there is little understanding of exactly how the microbiome directly influences a person's health and/or disease state. This is due largely to a lack of complete functional characterization of the human microbiome and resulting metabolome, which, though inextricably linked, are rarely analyzed either simultaneously or in terms of one another.

This, understandably, prevents microbiome and metabolome data from being used in several fields where it otherwise could prove useful, including direct-to-consumer knowledge, for example, home kits which assess microbiome health, and clinical applications, such as patient classification, diagnosis, and therapeutic predictions.

Despite the prominent role that the microbiome (and its corresponding metabolome) plays in individual health, our current lack of understanding of its components and functionality has prevented it from being of much use in clinical applications. Accordingly, a need arises for techniques that may profile and quantify the microbiome and metabolome and identify the novel health-related proteins that they affect.

### SUMMARY OF THE INVENTION

Embodiments of the present systems and methods may provide techniques that may profile and quantify the microbiome and metabolome and identify the health-related proteins that they affect. Embodiments may provide the capability for the classification of patients or other biological entities into clinical groups and labels, based on assessment of their microbiome and metabolome. Embodiments may provide the capability to assess patient health, identify disease risk factors, identify, and rank therapeutic targets, determine the functional contributions of the microbiome to patient health, and even predict outcomes such as disease development and drug response. For example, embodiments may include systems and methods that provide the capability to profile and quantify the microbiome and metabolome and identify the novel health-related proteins that they affect.

For example, in an embodiment, computer-implemented method for determining health-related features of microbes and metabolites may comprise receiving data obtained by collecting biological samples of material from a person and performing quantitative and qualitative physical analysis on the biological samples to generate data identifying species of microbes and metabolites in the biological samples, annotating and quantifying the data identifying species of microbes and metabolites, extracting features from the data identifying species of microbes and metabolites, determining a relative importance of the extracted features using a deep neural network, generating, using the extracted features and the relative importance of the extracted features, and by searching a protein-protein metabolite interactome (PPMI) in conjunction with data driven causal network-based approaches (for example, Bayesian Networks) to determine proteins that could be altered in the subject the sample was procured from, imputing clinical relevance to proteins present or interacting with the metabolite and microbe samples, determining a degree of centrality and a degree of betweenness of the imputed proteins, and determining a health related influence of each of at least some features, along with causal inference between microbe, metabolite, protein, and clinical features.

In embodiments, the biological samples may be selected from the group consisting of fecal samples, skin samples, tissue biopsies, urine, saliva, sputum, mucus, cerebrospinal fluid, and biofilm. Performing quantitative and qualitative physical analysis on the biological sample may comprise 16s rRNA sequencing or LC/MS. The method may further comprise obtaining clinical and lifestyle information from the subject. The clinical and lifestyle information may be selected from the group comprising age, sex, ethnicity, disease status, weight, diet, drug use, or a combination thereof.

In an embodiment, a method for determining health-related features of microbes and metabolites may comprise obtaining a biological sample from a subject, identifying and quantifying the species of microbes and metabolites in the biological sample, ranking the microbes and metabolites based on relative importance, and determining interactions between ranked microbes and metabolites and proteins to identify proteins involved in a health, lifestyle, or environmental-related condition.

In embodiments, ranking the microbes and metabolites may comprise using a deep neural network. Determining interactions between ranked microbes and metabolites and proteins may comprise using a protein-protein metabolite interactome and a microbe-metabolite interactome, and data driven causal connections. Identifying and quantifying the species of microbes and metabolites in the biological sample may comprise 16s rRNA sequencing or LC/MS. The biological samples may be selected from the group consisting of soil samples, fecal samples, skin samples, tissue biopsies, urine, saliva, sputum, mucus, cerebrospinal fluid, and biofilm.

In an embodiment, a system may comprise a processor, memory accessible by the processor, and computer program instructions stored in the memory and executable by the processor to perform receiving data identifying species of microbes and metabolites in a biological sample, the data generated by: obtaining a biological sample from a subject and performing quantitative and qualitative physical analysis on the biological sample to generate data, annotating and quantifying the data identifying species of microbes and metabolites, extracting features from the data identifying species of microbes and metabolites, determining a relative importance of the extracted features using a deep neural network, generating, using the extracted features and the relative importance of the extracted features, a subnetwork of proteins, metabolites, and microbes by searching a protein-protein metabolite interactome and a microbe-metabolite interactome or using a data driven causal network approach to determine proteins that could be altered in the subject the sample was procured from, imputing clinical relevance to proteins, metabolites, and microbes present or interacting with the metabolite and microbe samples, determining a degree of centrality and a degree of betweenness of the imputed proteins, metabolites, and microbes, and determining a health related influence of each of at least some features.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary flow diagram of an embodiment of a methodological process according to embodiments of the present systems and methods.

FIG. 2 illustrates an exemplary flow diagram of an embodiment of a methodological process according to embodiments of the present systems and methods.

FIGS. 3a, 3b, 3c show an exemplary flow diagram of a process, which may implement embodiments of the present methods, and which may be implemented in embodiments of the present systems.

FIGS. 4a, 4b, 4c, 4d illustrate an exemplary exploratory analysis of clinical samples for age and BMI matched control and colorectal cancer (CRC) patients using embodiments of the present systems and methods.

FIG. 5 illustrates an example of principal component analysis (PCA) according to embodiments of the present systems and methods.

FIG. 6 illustrates an example of a deep neural network according to embodiments of the present systems and methods.

FIGS. 7a, 7b, 7c illustrate an exemplary test of model accuracy using embodiments of the present systems and methods.

FIG. 8 illustrates an example of identification of metabolites according to embodiments of the present systems and methods.

FIG. 9 illustrates an example of network-based integration of microbiome and metabolome data, and inference of novel proteins according to embodiments of the present systems and methods.

FIG. 10 illustrates an example of GO enrichment analysis of inferred proteins from the network analysis shown in FIG. 9, according to embodiments of the present systems and methods.

FIG. 11 illustrates an example of identification of highly influential hub nodes according to embodiments of the present systems and methods.

FIG. 12 illustrates an exemplary flow diagram of a process of feature influence scoring according to embodiments of the present systems and methods.

FIG. 13 is an exemplary block diagram of a computer system in which processes involved in the embodiments described herein may be implemented.

### DETAILED DESCRIPTION

### Definitions

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “microbiota” refers to the ecological community of commensal, symbiotic, and pathogenic microorganisms that are found in and on a host. In humans, specific clusters of microbiota are found on the skin, or in the gastrointestinal tract, mouth, vagina, nasal passage, and eyes.

As used herein, “microbiome” refers to the full collection of genes of all the microbes in a community.

As used herein, “metabolites” refer to intermediate products of metabolic reactions that naturally occur within cells. They are the result of both biological and environmental factors. Metabolites are typically small molecules. “Metabolome” refers to the total number of metabolites present within an organism, cell, or tissue. “Metabolomics” is the comprehensive study of the low molecular weight molecules within an organism.

System and Methods for Classifying Health-Related Features

Embodiments of the present systems and methods may provide techniques that may profile and quantify the microbiome and metabolome and identify the novel health-related proteins that they affect. Embodiments may provide the capability for the classification of patients or other biological entities into clinical groups and labels, based on assessment of their microbiome and metabolome. Embodiments may provide the capability to assess patient health, identify disease risk factors, identify, and rank therapeutic targets, determine the functional contributions of the microbiome to patient health, and even predict outcomes such as disease development and drug response.

Embodiments of the present systems and methods may include a kit that may be sent to customers/patients for sample collection and returned to a suitable facility for processing via liquid chromatography mass spectroscopy (LC/MS) and 16s sequencing.

In some embodiments, in addition to the biological sample provided by patients, personal information such as age, sex, ethnicity, disease status, weight, diet, and current drug use may be collected, for example, via a cell phone application or website and may be used for individualized analysis.

In embodiments, 16s sequencing, which is a common method for bacterial identification, may be used to quantify and characterize the species of bacteria in the sample. Additionally, untargeted metabolomic profiling may be used to quantify and characterize the corresponding metabolites. In this way, both the microbiome and metabolome from the patient may be assessed.

In some embodiments, descriptive and exploratory statistics may be deployed on the patient data in order to prepare a comprehensive taxonomic and metabolic report. For example, in some embodiments, deep machine learning (ML) methods may be used for this analysis. ML processes may be applied to integrate the microbiome and metabolome data in order to identify functionally relevant species of microbes and metabolite levels with lifestyle and clinical features. Each data type (16s and metabolite) may be used independently and then combined for ML using a deep neural network to classify unique clinical labels (disease status, health outcome, lifestyle) with specific microbiome and metabolite signatures. These signatures may then be rank ordered for importance to clinical classification.

In some embodiments, identification of novel health-related proteins may be performed. Metabolomic data from the patient, which reveals which metabolites are present in the metabolome, may be used to predict the proteins that are most likely to interact and play a functional role in the clinical feature, using a network-based methodology. These imputed proteins may then be used for further analysis by generating networks based on the microbial, metabolite, and protein-protein interactions, and data driven directed acyclic causal networks. In embodiments, network analysis feature influence scoring may then be used to rank all species and molecules in the network for the largest biological influence towards the other network members and clinical feature being analyzed.

In embodiments, this analysis may provide for the complete assessment of a patient's microbiome (with associated metabolome and proteins), which, via deep ML assessment, may provide characterization of health status and even prediction of drug and disease outcomes.

Embodiments of the present systems and methods may provide comprehensive simultaneous analysis of the microbiome and metabolome. Microbiome data may be collected from a number of patient sites (oral, vaginal, gut, etc.) easily and painlessly. In embodiments, a kit can be deployed either directly to the consumer, via an at-home test, or dispatched via clinical settings. Based on the assessment of an individual's microbiome, embodiments may provide the capability for the prediction of future outcomes, including disease state, responsiveness to certain drugs, etc., and identification and ranking therapeutic targets.

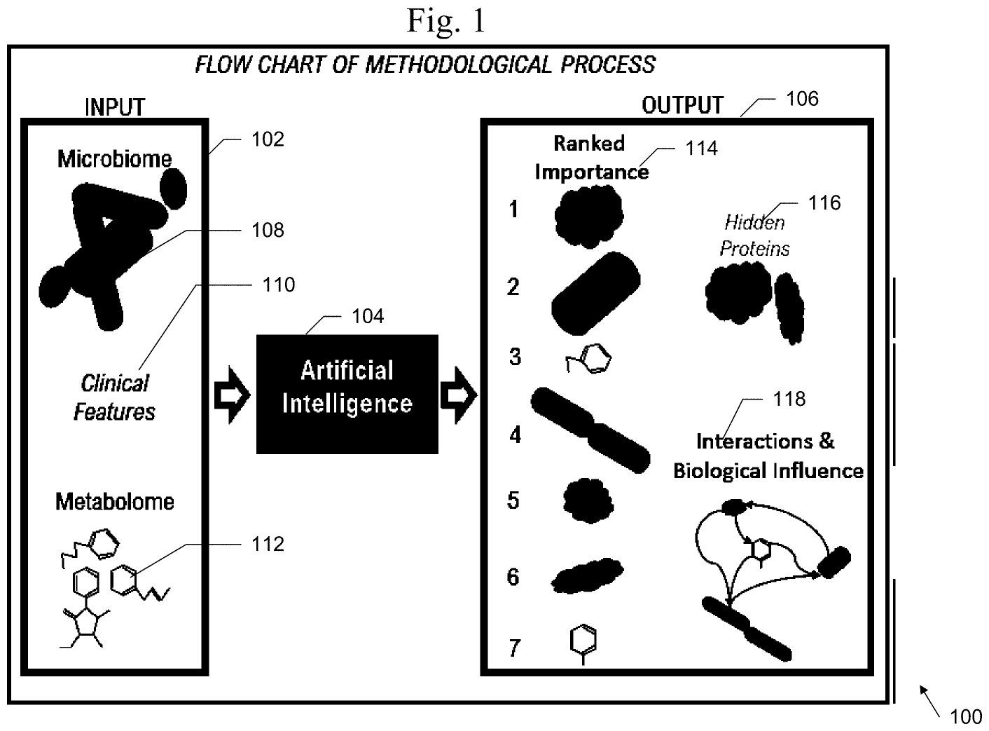

An exemplary flow diagram of an embodiment of a methodological process 100 is shown in FIG. 1. As shown in this example, an input 102 may be processed by, for example, artificial intelligence 104 to form an output 106. Input 102 may include data relating to the microbiome 108 of a subject, clinical features 110 of the subject, and the metabolome 112 of the subject. Output 106 may include a ranking 114 of microbiome and metabolite signatures, identifications 116 of hidden proteins, and identification of interactions and biological influence of the identified microbes and metabolites.

Artificial intelligence 104 may, for example, include deep machine learning to extract hidden data from input 102, such as a single biological sample. A network-based methodology may be used for discovery of novel health related proteins inferred from microbiome and metabolome. Artificial intelligence 104 may, for example, include finding and extracting important features and further using those for identifying hidden disease related proteins. In embodiments, a feature influence scoring process may be used to calculate the influence each molecule or species has on personal health/biology.

Embodiments of the present systems and methods may be applicable to a variety of uses, such as those shown in Table 1:

TABLE 1
Exemplary Uses of Disclosed Systems and Methods

| Ranked | Hidden | Interactions & Biological | |
|---|---|---|---|
| Importance | Proteins | influence | Other |
| Know what is | Find protein | Know how top targets | Classify any |
| important with | associations | interact | clinical, health, or |
| ranked correlation | without | | lifestyle related |
| against clinical | measuring | | metric |
| features | | | |
| Identify potential | ID novel | Uncover influence of | Disease diagnosis |
| therapeutics | disease- | microbes & metabolites on | & prognosis |
| | related | health and lifestyle | |
| | proteins | | |
| Narrow down | | Understand contributions | |
| target list for use in | | of microbes & how they | |
| biological | | confound treatment & | |
| investigation | | disease outcomes | |
| e.g. Network | | | |
| Analysis | | | |

Embodiments may provide the capability to perform deep machine learning to extract hidden data from a single biological sample, to perform network-based discovery of novel health related proteins inferred from microbiome and metabolome, to use artificial intelligence (AI) to find and extract important features and further use those features for identifying hidden disease related proteins, to perform feature influence scoring to calculate the influence each molecule or species has on personal health/biology.

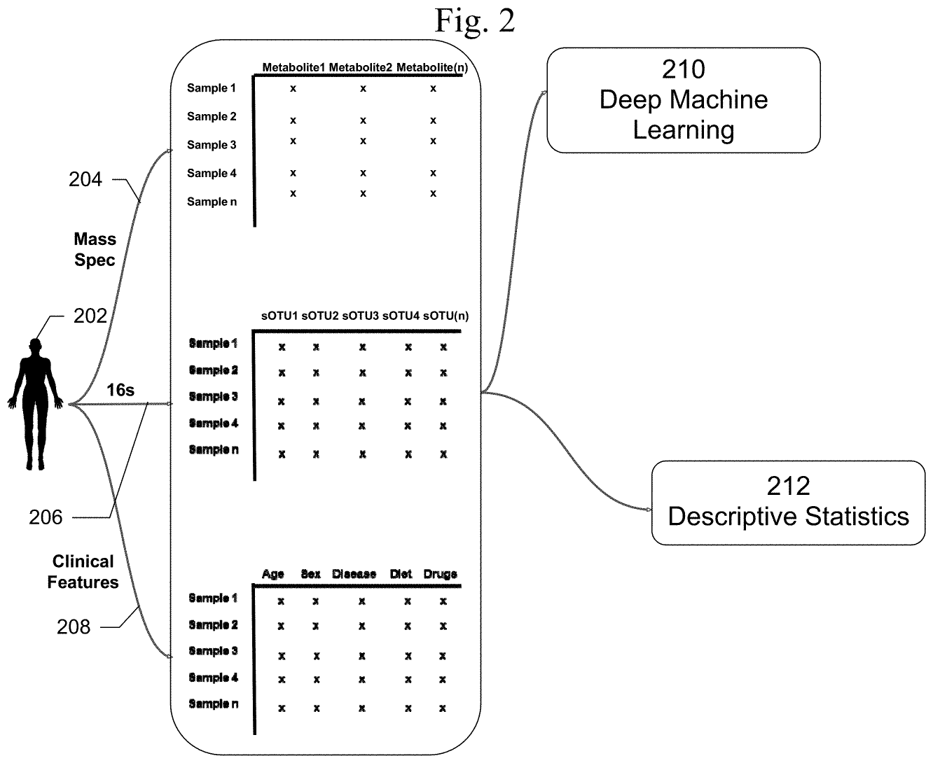

An exemplary flow diagram of an embodiment of a methodological process 200 is shown in FIG. 2. As shown in this example, samples and information may be obtained from one or more human subjects 202. The samples may be analyzed in a plurality of ways, such as mass spectrometry, etc. 204, mitochondrially encoded 16s RNA sequencing (16s) 206, etc. Further, information from a patent or clinician may be analyzed to extract clinical features 208. For example, mass spectrometry 204 of samples may provide information about metabolites present in the samples. 16s sequencing 206 may provide information about microorganisms at the phylum, class, order, family, genus, and species level. Likewise, clinical features that may be collected may include parameters such as age, sex, disease conditions, diet, and drug usage of the human subjects 202. The data resulting from the mass spectrometry 204, 16s sequencing 206, and clinical features collection 208 may be used to perform deep machine learning 210, to find patterns in the data, and may be analyzed using descriptive statistics 212, to provide human understandable information about the data.

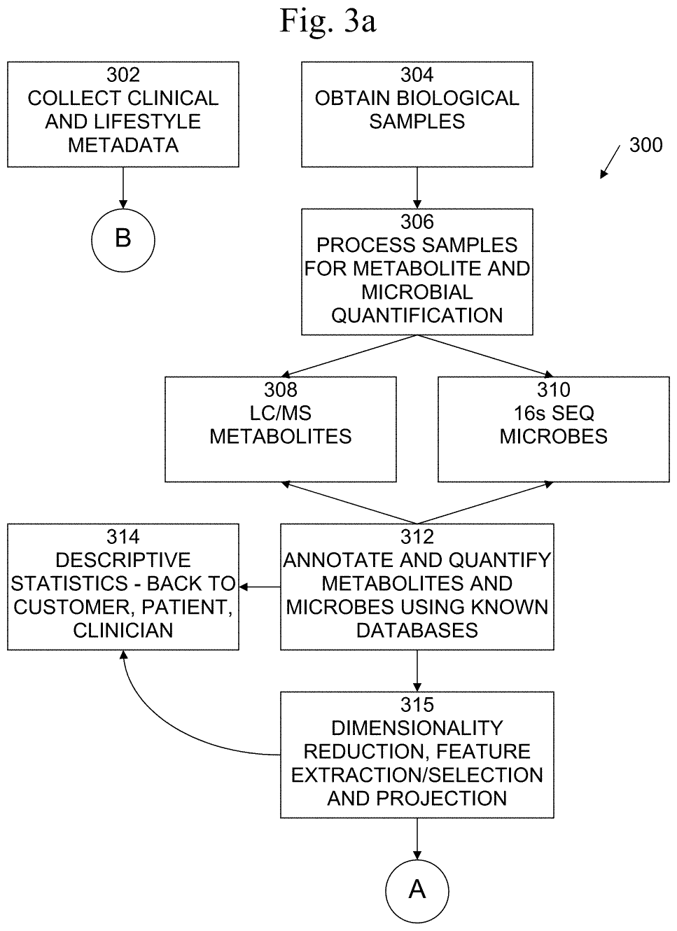

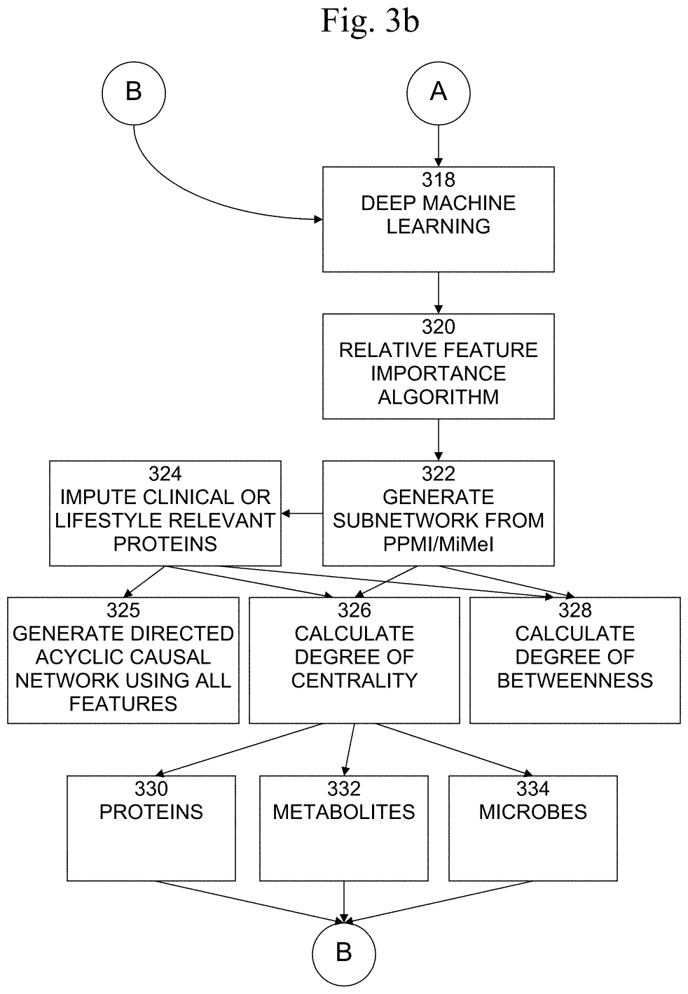

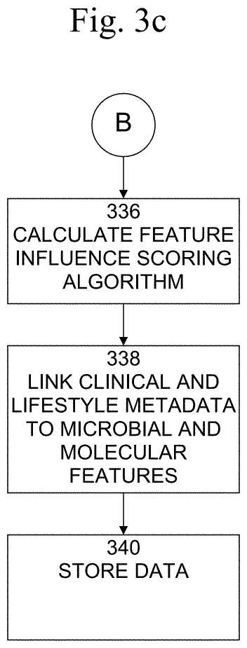

An exemplary flow diagram of an embodiment of a process 300 is shown in FIGS. 3a, 3b, 3c. Process 300 begins with 302, shown in FIG. 3a, in which clinical and lifestyle metadata may be collected. For example, clinical information may include but is not limited to patient information, such as age, sex, weight, height, disease conditions, clinical test results, and prescription drugs, while lifestyle information may include but is not limited to diet, exercise, and recreational drug use. This information may be formatted as metadata to accompany biological sample analysis data. At 304, biological samples may be obtained. At 306, the obtained samples may be processed for metabolite and microbial quantification. At 308, chemical analysis, such as liquid chromatography with tandem mass spectrometry may be performed on the processed metabolite samples to identify and quantify metabolites that may be present in the samples. At 310, mitochondrially encoded 16s RNA sequencing (16s) may be performed on the sample to identify microbe species.

At 312, the results of the chemical analysis and sequencing may be used to annotate and quantify the metabolites and microbes using, for example, known databases. The workflow for annotation and quantification of microbial species is as follows: 1) amplicon sequences from the variable regions of the 16s gene are collected for each sample, 2) sequences are then clustered into one of the typical units of measure using sequence similarity or de novo sequence clustering, e.g. operational taxonomic units (OTUs) or amplicon sequence variants (ASVs), 3) ASVs or OTUs are then assigned to taxa based on sequence annotation or sequence classification. The workflow for identification and relative quantification of metabolites is as follows: 1) Affinity purification using LC/GC-MS methods to obtain metabolite mass-to-charge ratios (m/z) and retention times, 2) differential metabolite peaks between clinical/lifestyle groups can be identified and further explored using tandem mass spectrometry to further resolve metabolites that are differential at those peaks, 3) these further resolved spectral libraries are then mapped to one of more databases on known metabolite spectral patterns. For example, for metabolites, the Human Metabolome Database (HMDB) (www.hmdb.ca) may be utilized, and for microbes the GreenGenes (greengenes.secondgenome.com) may be utilized. At 314, descriptive statistics characterizing and/or summarizing the metabolites and microbes present in the samples may be generated. Such statistics may include, for example, metabolites present and their quantities or concentrations, species and varieties of microbes present and their quantities or concentrations, locations of origin, etc. At 315, in embodiments, dimensionality reduction, feature extraction/selection, and projection may be performed. Such processes may include exploratory analysis and feature extraction, such as narrowing down all the features to only the ones that show high variance amongst patient groups, using unsupervised learning techniques, such as principal component analysis (PCA), Linear discriminant analysis (LDA), canonical correlation analysis (CCA), singular value decomposition (SVD), or other similar linear or non-linear dimensionality reduction methods. The results from 315 may be provided to descriptive statistics processing 314, as well as to deep machine learning 316.

At 316, the annotated and quantified information relating to the metabolites and microbes, along with the corresponding clinical and lifestyle metadata for the patient may be input to a deep machine learning process for analysis. For example, in embodiments, the deep machine learning processing may include an artificial neural network with many neuron layers. In embodiments, the deep machine learning processing may include other machine learning techniques, such as logistic and linear regression analysis, support vector machines, naïve Bayes, Bayesian networks, decision tree learning (e.g. random forests), or other statistical classification methods.

Turning now to FIG. 3b, at 318, the results from the deep machine learning process may be input to a deep neural network for feature extraction and identification. At 320, the relative importance of the identified features may be determined, for example, by techniques such as by the “Connection weights” algorithm (Olden's algorithm), Garson's algorithm, perturbation feature ranking algorithms, or other similar techniques. At 322, a subnetwork may be generated by searching a protein-protein metabolite interactome (PPMI) and/or microbe-metabolite interactome (MiMel) to determine connections between ranked metabolite and microbial features and proteins that could be altered in the patient the sample was procured from. Metabolites and/or microbes identified as top ranked features are treated as nodes and a single edge growth between these nodes and hidden proteins are connected based on a curated list of all known and direct protein-protein, protein-metabolite, or metabolite-microbe interactions. An iterative process adding additional edge growths will be used to assess further connections between metabolites and microbes identified in patient samples, and any additional imputed hidden proteins. Biological and statistical assessment of each of these subsequently generated subnetworks with n number of edge growths will be used to determine an appropriate n. After each additional edge growth all nodes with in the subnetwork will be used for biological enrichment and overrepresentation analyses. Pathways, biological networks, and gene ontology analyses will be used to determine biological and statistical significance of the subnetwork, a hypergeometric or similar test will be used to calculate enrichment or overrepresentation of biological relevant terms. The iterative process will continue until 1) no additional edges/nodes can be added to the subnetwork, 2) interpretable biological enrichment is obtained with at least 1 statistically significant biological term, 3) adding any additional nodes reduces the number of statistically significant biological terms or leaves the subnetwork uninterpretable. All iterations representing each subsequent subnetwork may be used for analyses further downstream the invention workflow. At 324, the subnetwork may be used to impute clinical and/or lifestyle relevance to proteins present or interacting with the metabolite and microbe samples. At 325, all nodes from previous subnetwork and clinical features may be used for network-based causal inference analysis (e.g. Bayesian network analysis) to generate a directed acyclic graphical representation of the subnetwork from 322 and 324 and to link measured biological features with clinical or lifestyle data.

At 326, the degree of centrality of the imputed proteins may be determined using the subnetwork. The degree of centrality of each hidden protein is calculated as the number of neighbor nodes, connected by an edge, the protein has divided by the total number of potential neighbor nodes. Likewise, at 328, the degree of betweenness of the imputed proteins may be determined using the subnetwork. The betweenness centrality of each hidden protein is calculated as the number of shortest paths through the protein node divided by all possible shortest paths. At 330, 332, and 334, the proteins, metabolites, and microbes, respectively, are determined. At 336, the influence of the features, that is, the proteins, metabolites, and microbes, may be determined using a scoring process as described below. At 338, the clinical and lifestyle metadata may be linked to microbial and molecular features. For example, the features may be identified and ranked by adding in the clinical and lifestyle metadata during the machine learning process, as in 325 where data driven causal connections will link these features with the metadata, so they may be identified as important and may be linked to the metadata by the machine learning process itself. In addition, to further link the importance of the individual features, the role of each feature may be assessed biologically to that metadata. For example, if HER2 was identified from the process, and its role in breast cancer wasn't known, the next step would be to assess how it would be relevant to breast cancer. At 340, the generated data may be stored.

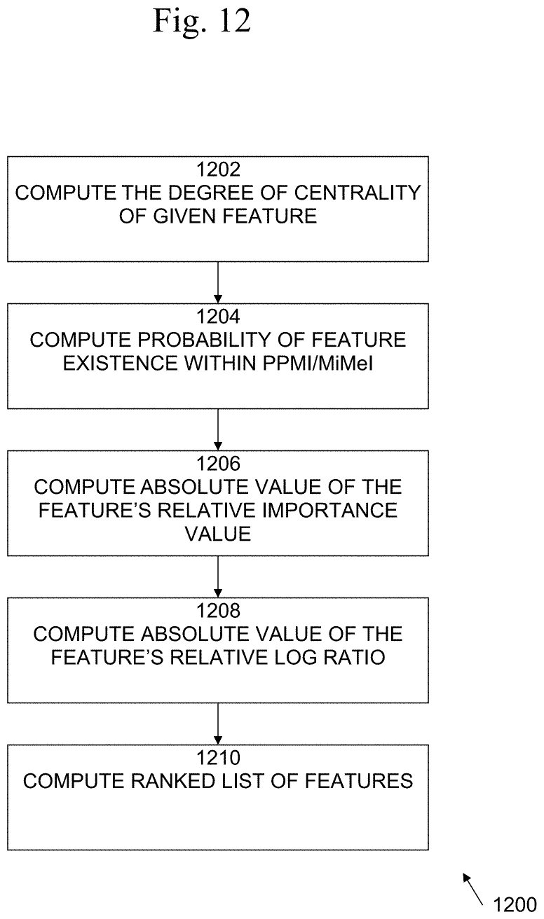

An exemplary flow diagram of a process 1200 of feature influence scoring, such as may be performed at 336 of FIG. 3c, is shown in FIG. 12. 1202-1208 may be repeated for each feature in a subnetwork (g), as specified in a list L. Process 1200 may begin with 1202, in which the degree of centrality of a given feature A may be computed. At 1204 the probability that a feature B exists in g, given the PPMI network G currently in use may be computed as P(B|G). At 1206, the absolute value of the relative importance of the feature may be computed as |C|. At 1208, the absolute value of the relative log ratio of the feature may be computed as |D|. At 1210, a ranked list of numerical values M for all features given values in L may be computed as

$$M = ( \frac{A}{P ( B \vert G )} ) \vert C \vert \vert D \vert$$

Sample Collection and Processing

Samples to be analyzed using the disclosed systems and methods can be collected from a variety of anatomical locations, including but not limited to the mouth, nose, gastrointestinal tract, vagina, skin, nasal cavities, ears, and lungs. Samples can be collected from many types of tissues by swabbing the tissue. Exemplary tissue that can be analyzed via swabbing include but are not limited to skin, buccal mucosa (cheek), gums, palate, tonsils, throat, tongue, tooth biofilm above and below the gum line, within the nose, rectum, and vagina. Biofluids such as urine, plasma, saliva, sputum, mucus, and CSF can also be collected and analyzed. In addition, fecal samples, skin samples, and tissue biopsies or homogenates can also be used for testing. Samples can be collected by non-invasive or semi-invasive means.

In one embodiment, a subject is provided a container in which to collect or deposit the sample. The container can be any suitable vessel for holding the sample. Exemplary containers include but are not limited to a vial, tube, bag, sample-chamber, well-plate, or any other suitable sample container.

In some embodiments, the subject produces or procures the sample outside of a hospital or clinic setting. In such embodiments, the subject can be provided materials and instructions for sending the sample to a clinic or lab. In other embodiments, the sample is collected from the subject at a lab or clinic.

After sample procurement, the samples can be processed for microbial and metabolic profiling and quantification. The samples can be processed for 16s sequencing or metabolomic profiling. In some embodiments, the samples can be collected directly into buffers necessary for processing, such as but not limited to PCR buffers, PBS, methanol, or any other appropriate liquids.

In one embodiment, samples are processed for 16s rRNA sequencing. 16s sequencing is a common amplicon sequencing method used to identify and compare bacteria present within a given sample. The sequences of the 16s RNA gene contain hypervariable regions which can provide specific signature sequences useful for bacterial identification. It can provide characterization of microorganisms at the phylum, class, order, family, genus, and species level. 16s sequencing data can be used to quantify and characterize the species of bacteria using standard data processing including sequencing read QC, alignment, and quantification.

16s rRNA sequencing methods are known in the art. See for example, Tremblay, et al., Front Micrbiol, 6:771 (2015); Clarridge, et al., Clin Microbiol Rev, 17:840-862 (2004)). DNA can be extracted from the samples using commercially available kits, for example PowerSoil® DNA Isolation Kit (Mo Bio Laboratories, Carlsbad, Calif.). 16s rRNA can be detected by various amplicons, for example amplicons covering variable regions V3 to V4 of the 16s sequence. Amplicons can be sequenced using various means, for example 454 Roche FLX Titanium pyrosequencing system.

Other methods of detecting microbes in a sample include but are not limited to PCR amplification by degenerate primers, tblastn analysis, and microbial physiology.

In another embodiment, samples are processed for metabolomic profiling. A variety of separation methods can be used for metabolomics experiments, including but not limited to high-performance liquid phase chromatography (HPLC), gas chromatography (GC), and capillary electrophoresis (CE), or a combination thereof. Metabolite extraction can be performed using various techniques known in the art, for example non-targeted methanol extraction and protein precipitation.

The two main detection methods used for metabolomics experiments include but are not limited to nuclear magnetic resonance (NMR) and mass spectrometry (MS), both of which allow for the detection of many different metabolites. In a preferred embodiment, the method of detecting metabolites in a sample can be HPLC-GC/MS-MS. Individual molecules and their relative levels can be identified from the mass spectral peaks compared to a reference library generated from standards, based on mass spectral peaks, retention times, and mass-to-charge ratios. Molecules that can be identified include but are not limited to amino acids, carbohydrates, fatty acids, androgens, and xenobiotics.

After the samples have been processed, untargeted metabolomic profiling can be used to quantify and characterize all metabolites. Untargeted metabolomics provides a comprehensive analysis of all the measurable analytes in a sample including chemical unknowns. In another embodiment, targeted metabolomics profiling can be used to measure defined groups of chemically characterized and biochemically annotated metabolites.

### Gut Microbiome, Metabolome, and Related Diseases

The gastrointestinal tract is host to commensal and pathogenic microbes. Exemplary commensal bacteria in the gastrointestinal tract include but are not limited to, Bacteroides, Clostridium, Prevotella, Porphyromonas, Eubacterium, Ruminococcus, Streptococcus, Enterobacterium, Enterococcus, Lactobacillus, Peptostreptococcus, Fusobacteria, Lacnospira, Roseburia, and Butyrivibrio. Exemplary pathogenic gut bacteria include but are not limited to Campylobacter jejuni, Salmonella enterica, Vibrio cholera, Escherichia coli, and Bacteroides fragilis.

In one embodiment, the disclosed systems and methods can be used to determine the relative abundance of gut microbes in a subject. In another embodiment, the disclosed systems and methods can be used to detect microbes and/or metabolites that are involved in disease pathogenesis.

In the proximal GI tract, simple sugars such as glucose are absorbed, and disaccharides are hydrolyzed into their corresponding monosaccharide components such that they can be absorbed. A significant portion of dietary carbohydrates, including complex plant-derived polysaccharides and unhydrolyzed starch, normally passes undigested through to the distal GI tract. Microbes within the distal GI tract are well-equipped to hydrolyze complex carbohydrates. Short chain fatty acids (SCFAs) are metabolites produced from the fermentation of indigestible oligosaccharides, dietary plant polysaccharides or fibers, non-digested proteins, and intestinal mucin. SCFAs include but are not limited to butyrate, acetate, and propionate. The colonic epithelium derives up to 70% of its energy needs directly from butyrate. It is believed that SCFAs also impact water absorption, local blood flow, and epithelial proliferation in the large intestine.

SCFAs are produced by clostridial clusters IV, XIVa (which include but are not limited to Eubacterium, Roseburia, Faecalibacterium, and Coprococcus sp.), Lactobacillus, and the family of Actinobacteria (Bifidobacterium spp.).

In one embodiment, the lack of SCFA producing bacteria can indicate disease in a subject. In another embodiment, the lack of SCFAs can indicate disease in a subject. Exemplary diseases related to a lack of SCFA in the gut include but are not limited to diversion colitis, ulcerative colitis, other inflammatory diseases, and colorectal cancer.

Conventional knowledge suggests that all essential amino acids can be derived by diet. However, studies indicate that the intestinal microbiota makes a measurable contribution to the pool of essential amino acids. Amino acids, peptides, fatty acids, sugars, and other organic compounds that may be produced by bacteria in the gut include but are not limited to lysine, threonine, citrulline, phenylacetate, glutamate, cysteine, indolepropionate, N-formylmethionine, cadaverine, phenethylamine, 2-hydroxybutyrate, homoserine, N-acetylglutamine, N-methylphenylalanine, glutaminylisoleucine, glutamyltryptophan, aspartylphenylalanine, isoleucyl-glycine, isoleucyl-isoleucine, isoleucyl-serine, isoleucyl-valine, threonyl-isoleucine, serylleucine, N-acetylalanine, N-acetylarginine, 2-aminobutyrate, creatinine fructose, galactose, glutamate, and glucose.

Bacteria involved in the production of amino acids include but are not limited to Clostridia, Peptostreprococcus anaerobius, Streptococcus bovis, Selenomonas ruminantium, and Prevotella bryantii.

In one embodiment, the presence of microbiota involved in the production of amino acids can indicate disease. In another embodiment, the absence of microbiota involved in the production of amino acids can indicate disease. Comparative levels of choline, trimethylamine N-oxide (TMAO), and betaine, three metabolites of dietary phosphatidylcholine, can be used to predict cardiovascular disease risk in subjects.

Organic acids result from bacterial metabolism of dietary polyphenols or unassimilated amino acids or carbohydrates. Organic acids have been associated with hypertension, obesity, colorectal cancer, and diabetes. Organic acids include but are not limited to benzoate, fumarate, hippurate, phenylacetate, phenylpropionate, hydroxybenzoate, N-2,acetyl lysine, 4-acetamidophenol, Alanyl isoleucine, Alanyl valine, hydroxyphenylacetate, dihydroferulic acid, 2-aminoadipate, N-acetylmuramate, arachidic acid, taurine, dihydrocaffeic acid, pyridoxate, 2-hydroxydecanoic acid, kynurenate, 3-hydroxydecanoate, 8-hydroxyoctanoate, hydroxylphenylpropionate, daidzein, 3-hydroxypyridine, 3,4-dihydroxyphenylpropionate, mandelate, tryptophyl-valinepterin, valyl-isoleucine, valyl-valine, 3,7-dimethylurate, 7-methylguanine, 6-hydroxynicotinate, 6-oxopiperidine-2-carboxylic acid, tricarballylate, 3-(3-Hydroxyphenyl)propanoic acid, hydroxypropionic acid, 1,3,7-trimethylurate, tyrosol, p-Aminobenzoic acid, phenyllactic acid, dihydroferulic acid, quinate, xanthine, p-cresol sulfate, 7-methylguanine, indoleacetate, L-allothreonine and D-lactate.

Bacteria involved in the production of organic acids include but are not limited to Clostridium difficile, Faecalibacterium prausnitzii, Bifidobacterium, Subdoligranulum, and Lactobacillus.

While the majority of vitamins required by humans can be obtained through diet alone, gut microbes also contribute to vitamin synthesis. Vitamins produced by bacteria in the gut include but are not limited to niacin, pyridoxal, nicotinate, arabonate, threonate, pantothenate, thiamine, folate, biotin, riboflavin, pyridoxal, Vitamin K, and panthothenic acid. Bacteria involved in the production of vitamins include but are not limited to Bifidobacterium bifidum, Bifidobacterium longum, Bifidobacterium breve, Bifidobacterium adolescentis, commensal Lactobacilli, Bacillus subtillis, Escherichia coli, Bacteroides, Enterococcus, Fushobacteria, Proteobacteria, and Actinobacteria.

Neuroactive metabolites, ranging from serotonin and gammaaminobutyric acid (GABA), to dopamine and norepinephrine, to acetylcholine and histamine tryptophan, serotonin, and indoles can be produced by gut microbes for example by the metabolism of monosodium glutamate. Exemplary microbes that produce neuroactive metabolites include but are not limited to Bifidobacteria and Lactobacillus spp. In one embodiment, detection of neuroactive metabolites in fecal samples can indicate disease.

Exemplary lipids produced by gut microbes include but are not limited to behenic acid, tetracosanoic acid, beta sitosterol, campesterol, Glycerol 3 phosphate, docosapentaenoate, isopalmitic acid, lithocholate, oleate, adipate, isocaproate, lanosterol, myristoleate, palmitoleate, squalene, glycocholate, 1-hexadecanol, 1-octadecanol, nervonic acid, 12-methyltridecanoic acid, Vaccenic acid, pentadecanoate, 1-palmitoylglycerol.

Nucleosides such as but not limited to guanosine, uridine, uracil, 2-deoxyguanosine, 2-deoxyuridine, cytidine, and pseudouridine.

Disruption of the normal equilibrium between a host and its gut microbiota is associated with a number of conditions and diseases in the gastrointestinal tract. In one embodiment, profiling and quantifying the microbiome and metabolome.

The microbial ecology of the GI tract has been shown to contribute to the pathogenesis of obesity. Decreased abundance of Bacteriodetes and increased abundance of Firmicutes is a characteristic of the gut microbiome of subjects with obesity. It is believed that this imbalance leads to improper lipid metabolism. Other microbes that have been implicated in the pathogenesis of obesity include but are not limited to Proteobacteria and Bifidobacterium spp. Microbial metabolites that have been implicated in the pathogenesis of obesity include but are not limited to hippurate, 4-hydroxyphenylacetic acid, phenlyacetylglycine, FFA, BCAA, primary bile acids such as cholic and chenodeoxycholic acid, and secondary bile acids such as lithocholic acid.

In one embodiment, the detection of Bacteroides and Firmicutes can indicate the pathogenesis of obesity. In another embodiment, the relative abundance of Bacteroides and Firmicutes in a subject is analyzed over time to monitor disease progression.

Inflammatory bowel disease (IBD) and irritable bowel syndrome (IBS) are often characterized by an abnormal composition of the gut microbiome. Subjects with IBD and IBS often show high levels of Proteobacteria and decreased levels of Actinobacteria and Firmicutes compared to healthy subjects. Clostridium clusters XIVa and IV have also been implicated in the pathogenesis of IBD/IBS. Irregular microbial fermentation leads to the high production of hydrogen, indoles, phenols, and other volatile organic compounds which cause a heightened immune response in the intestinal tissue.

Metabolites that have been implicated in the pathogenesis of IBS include but are not limited to hydrogen and esters. Metabolites involved in the pathogenesis of IBD include but are not limited to alcohols, esters, indoles, phenols, acetone, sulfur compounds, propanoic and butanoic acids, phenol and p-cresol, hippurate, tyrosine, dopamine, tryptophan, phenylalanine, isoleucine, leucine, lysine, bile acids, cadaverine, and taurine.

In some embodiments, increased concentrations of Bacterioides, Eubacteria, and Peptostreptococcus and decrease concentrations of Bifidobacteria are indicative of Crohn's disease. In another embodiment, increased concentrations of facultative anaerobes is indicative of ulcerative colitis. In such embodiments, the concentrations of microbiota and metabolites in a sample from a subject are compared to a microbiota and metabolite panel from a healthy subject or subjects. In another such embodiment, the concentration of microbiota and metabolites in a sample from a subject are compared to microbiota and metabolites from a sample that was previously collected from the same subject.

The human gut microbiome has been implicated in the pathogenesis of colorectal cancer. Pathogenic microbes such as Escherichia coli produce toxins including colibactin and cytolethal distending toxin that can induce DNA damage and the progression of CRC. Enterococcus faecalis has been shown to produce extracellular superoxide and hydrogen peroxide which damage DNA. Bacteria in cluster IX of the genus Clostridium spp. convert bile acids into a secondary bile acid such as deoxycholic acid which is a carcinogen.

Other microbiota that have been implicated in the formation or progression of CRC include but are not limited to, Fusobacterium nucleatum, Porphyromonas, Clostridium spp., Lachnospiracea, H. pylori, Acidovorax spp., Bacteroides fragilis, Streptococcus bovis, and Salmonella spp.

Exemplary metabolites that have been implicated in the pathogenesis of CRC include but are not limited to palmitoyl-sphingomyelin, p-hydroxyl-benzaldehyde, p-aminobenzoate, conjugated linoleic acid, mandelate, and alpha tocopherol.

In some embodiment, detection of the above-mentioned microbiota and metabolites implicated in the pathogenesis of colorectal cancer in a subject compared to the microbiome of a known healthy subject can indicate colorectal cancer. In another embodiment, alterations in the level of the above-mentioned microbiota and metabolites implicated in the pathogenesis of colorectal cancer can indicate progression or remission of colorectal cancer.

Gut microbes that have been correlated with cystic fibrosis include but are not limited to Pseudomonas aeruginosa, Clostridium clusters XIVa and IV, Clostridium acetobutylicum, F. prausnitzii, Eubacterium limnosum, Eubacterium biforme, E coli, and Bifidobacterium spp. Metabolites that are correlated with cystic fibrosis include but are not limited to C5-C16 hydrocarbons, N-methyl-2-methylpropylamine ethanol, methanol, acetate, 2-propanol, lactate, dimethyl sulfide, and acetone. Increased levels of 2,3-butanedione in the lungs can indicate cystic fibrosis. 2,3-butanedione is produced by Streptococcus spp.

In one embodiment, the disease progression of cystic fibrosis can be monitored by measuring the levels and relative abundance of any one of the following microbes, Pseudomonas aeruginosa, Clostridium clusters XIVa and IV, Clostridium acetobutylicum, F. prausnitzii, Eubacterium limnosum, Eubacterium biforme, E. coli, and Bifidobacterium spp.

Gut microbes that have been associated with non-alcoholic fatty liver disease (NAFLD) include but are not limited to Oscillospira, Rickenellaceae, Parabacteroides, Bacteroides fragilis, Sutterella, and Lachanospiraceae. Metabolites that have been implicated in the pathogenesis of NAFLD include but are not limited to ethanol, ester, 4-methyl-2-pentanoate, 1-butanol, and 2-butanoate.

Gut microbes that have been associated with Celiac disease include but are not limited to Lactobacillus, Enterococcus, Bifidobacteria, Bacteroides, Staphylococcus, Salmonella, Shigella, and Klebsiella. Metabolites implicated in the pathogenesis of Celiac disease include but are not limited to acetoacetate, glucose, 3-hydroxybutyric acid, indoxyl sulfate, meta-[hydroyphenyl] propionic acid, phenylacetylglycine, 1-octen-3-ol, ethanol, 1-propanol, amino acids such as proline, methionine, histidine, and tryptophan; choline, lactate, methylamine, ethyl acetate, and pyruvate.

Oral Microbiome, Metabolome, and Related Diseases

Microbiota commonly found in the oral cavity include but are not limited to, Streptococcus gordonii, Streptococcus mitis, Streptococcus oralis, Streptococcus salivarius, Actinomyces naeslundii, Veillonella, Fusobacterium nucleatum, Porphromonas, Prevotella gingivalis, Prevotella loescheii, Veillonella atypica, Treponema medium, Nisseria, Haemophilis, Eubacteria, Lactobacterium, Capnocytopha gingivalis, Capnocytophaga ochracea, Eikenella, Leptotrichia, Peptostreptococcus, Staphylococcus, and Propionibacterium.

Saccharolytic bacteria—including Streptococcus, Actinomyces, and Lactobacillus species—degrade carbohydrates into organic acids resulting in dental caries, while alkalization and acid neutralization via the arginine deiminase system and urease counteract acidification. Proteolytic/amino acid—degrading bacteria, including Prevotella and Porphyromonas species, break down proteins and peptides into amino acids and degrade them further via specific pathways to produce short-chain fatty acids, ammonia, sulfur compounds, and indole/skatole, which act as virulent and modifying factors in periodontitis and halitosis. Furthermore, it is suggested that ethanol-derived acetaldehyde can cause oral cancer, while nitrate-derived nitrite can aid caries prevention and systemic health. Chronic gingivitis and periodontitis are also thought to be caused by an imbalance in oral microbes.

Skin Microbiome, Metabolome, and Related Diseases

Exemplary skin microbiota include but are not limited to Staphylococcus epidermidis, Staphylococcus aureus, Staphylococcus warneri, Streptococcus pyogenes, Streptococcus mitis, Propionibacterium acnes, Corynebacterium spp., Acinetobacter johnsonii, and Pseudomonas aeruginosa. P. acnes hydrolyses the triglycerides present in sebum, releasing free fatty acids onto the skin.

Diseases of the skin that have been reported to be linked to microbial imbalance include but are not limited to sebborhoeic dermatitis, teenage malady acne, atopic dermatitis, wound infection and lack of healing, eczema, rosacea, psoriasis, and acne.

### Urogenital Microbiome, Metabolome, and Related Diseases

Microbiota commonly found in the urogenital tract include but are not limited to, Lactobacillus species L. crispatus, L. iners, L. gasseri and L. jensenii, Gardnerella vaginalis, Atopobium, Corynebacterium, Anaerococcus, Peptoniphilus, Prevotella, Gardnerella, Sneathia, Eggerthella, Mobiluncus, Mycoplasma hominis, Enterobacter and Finegoldia. An exemplary group of metabolites implicated in disease in the urogenital tract are thiopeptides.

Disruptions in homeostasis of urogenital microbiota can lead to diseases and disorders including but not limited to, symptomatic bacterial vaginosis, yeast infections, sexually transmitted infections (STI), and urinary tract infections.

System for Classifying Health-Related Features

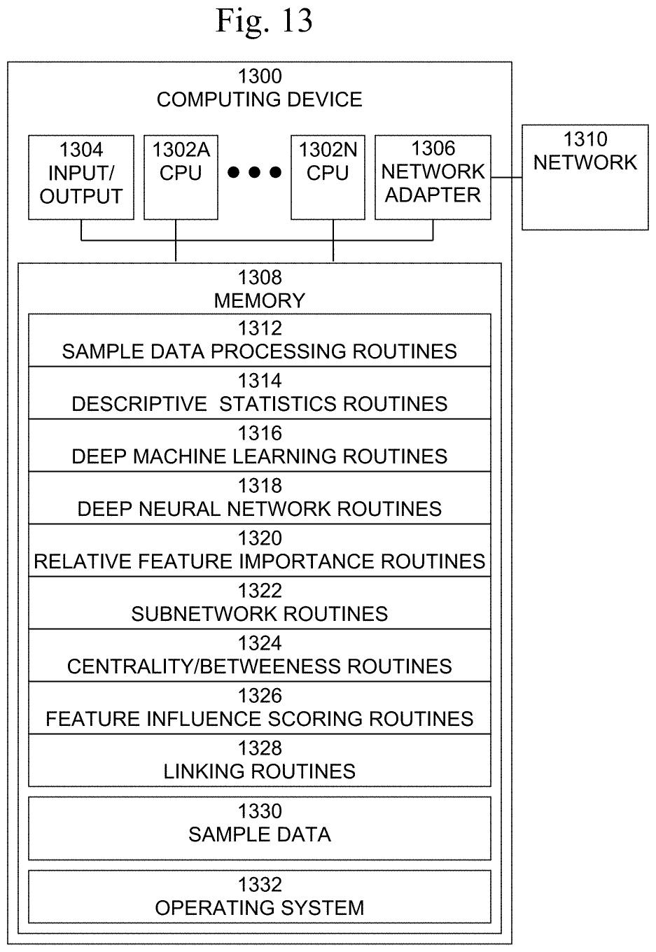

An exemplary block diagram of a computer system 1302, in which processes involved in the embodiments described herein may be implemented, is shown in FIG. 13. Computer system 1302 may be implemented using one or more programmed general-purpose computer systems, such as embedded processors, systems on a chip, personal computers, workstations, server systems, and minicomputers or mainframe computers, or in distributed, networked computing environments. Computer system 1302 may include one or more processors (CPUs) 1302A-1302N, input/output circuitry 1304, network adapter 1306, and memory 1308. CPUs 1302A-1302N execute program instructions in order to carry out the functions of the present communications systems and methods. Typically, CPUs 1302A-1302N are one or more microprocessors, such as an INTEL CORE® processor. FIG. 13 illustrates an embodiment in which computer system 1302 is implemented as a single multi-processor computer system, in which multiple processors 1302A-1302N share system resources, such as memory 1308, input/output circuitry 1304, and network adapter 1306. However, the present communications systems and methods also include embodiments in which computer system 1302 is implemented as a plurality of networked computer systems, which may be single-processor computer systems, multi-processor computer systems, or a mix thereof.

Input/output circuitry 1304 provides the capability to input data to, or output data from, computer system 1302. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 1306 interfaces device 1300 with a network 1310. Network 1310 may be any public or proprietary LAN or WAN, including, but not limited to the Internet.

Memory 1308 stores program instructions that are executed by, and data that are used and processed by, CPU 1302 to perform the functions of computer system 1302. Memory 1308 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 1308 may vary depending upon the function that computer system 1302 is programmed to perform. In the example shown in FIG. 13, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present systems and methods may include any and all such arrangements.

In the example shown in FIG. 13, memory 1308 may include sample data processing routines 1312, descriptive statistics routines 1314, deep machine learning routines 1316, deep neural network routines 1318, relative feature importance routines 1320, subnetwork routines 1322, centrality/betweenness routines 1324, feature influence scoring routines 1326, linking routines 1328, sample data 1330, and operating system 1320. Sample data processing routines 1312 may include software routines to process sample data 1330 relating to metabolite and microbial samples to prepare the data for further processing, as described above. Descriptive statistics routines 1314 may include software routines to generate descriptive statistics, as, for example, at 314 of FIG. 3. Deep machine learning routines 1316 may include software routines to perform deep machine learning, as, for example, at 316 of FIG. 3. Deep neural network routines 1318 may include software routines to perform deep neural network processing, as, for example, at 318 of FIG. 3. Relative feature importance routines 1320 may include software routines to determine relative feature performance, as, for example, at 320 of FIG. 3. Subnetwork routines 1322 may include software routines to generate subnetworks from PPMI, as, for example, at 322, or directed acyclic causal networks, as, for example, at 325 of FIG. 3. Centrality/betweenness routines 1324 may include software routines to determine degrees of centrality and betweenness, as, for example, at 326, 328 of FIG. 3. Feature influence scoring routines 1326 may include software routines to determine feature influence scores and rankings, as, for example, at 336 of FIG. 3 and 1200 of FIG. 12. Linking routines 1328 may include software routines to link clinical and lifestyle metadata to microbial and molecular features, as, for example, at 338 of FIG. 3. Operating system 1320 may provide overall system functionality.

As shown in FIG. 13, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Methods of Use

The disclosed systems and methods can be used for profiling and quantifying the microbiome and metabolome of a subject. In addition, the disclosed systems and methods can be used for example to determine the ranked importance of biological targets, to find hidden proteins that could be potentially important to disease, to identify interactions between biological targets and how they influence health and lifestyle, and as a diagnostic tool.

The disclosed systems and methods can be used to obtain a complete report of a subject's microbiome and metabolome from a single sample. The report can be used for example to assess patient health or predict disease progression, to assess drug response, or to rank therapeutic targets. In one embodiment, the specific microbiota and metabolites in the patient sample are indicative of a specific disease or disorder. Diseases can exhibit the presence of a novel microbe, the absence of a normal microbe, or an alteration in the proportion of microbes. In addition, the production of certain metabolites can cause or exacerbate diseases or disease phenotypes. The disclosed systems and methods can be used to diagnose or monitor the progression of disease.

Embodiments of the present systems and methods may provide the capability for, for example, profiling microbes and metabolites for classification of patients or other biological entities into clinical groups, for identification of health-related proteins, and for the identification of therapeutic targets. Embodiments of the present systems and methods may provide simultaneous analysis of the microbiome and metabolome. Microbiome data may be collected from patient sites (oral, vaginal, gut, etc.) easily and painlessly. The collection apparatus may be offered as a kit that may be used either directly by consumers via an at-home test or dispatched via clinical settings. Microbiome and metabolome characterization may rely on standard, pre-established techniques.

Embodiments of the present systems and methods may provide the capability for, for example, the prediction of future outcomes, including a patient's disease state, responsiveness to certain drugs, etc., for overall health to determine nutrition plans, for precision medicine to assess if certain medications will work well/at all for certain individuals, for pharmacological assessment to determine if pharmaceuticals that individuals are taking are having desired effect or adverse side effects, for toxicology studies to assess how LD50 or lower/higher levels of chemicals affect imputed features based on combined microbiome and metabolome in small mammal studies. Embodiments of the present systems and methods may provide the capability for, for example, veterinary pathology to assess disease diagnosis in pets and livestock, and for agriculture to determine how various feeds affect whole animal function and the effect of soil microecology and metabolite composition on crop quality and yield. Embodiments of the present systems and methods may provide the capability for, for example, medical applications to determine the effect medical foods, such as bacterial therapeutics, are having on protein composition and metabolite production as microbial communities change with therapeutic use, which may become relevant as bacteria move away from the supplement space and into the medical food category. Embodiments of the present systems and methods may provide the capability for, for example, child care to analyze samples from newborns and correlate to clinical metadata, to determine how clinical features have affected microbiome and metabolome, and consequently, protein production, and to customize formula supplements to alter protein production in vulnerable infants (C-section, antibiotic use after birth, etc.) to more closely match non-vulnerable infants (vaginal birth, breastfed consistently, etc.). Embodiments of the present systems and methods may provide the capability for, for example, biomarkers/biosensors to monitor health and assess health over time, and may be applied to other data types or in combination, for genetic, RNA, and protein analysis, and for collaboration with genetic assessment companies. Embodiments of the present systems and methods may provide the capability for, for example, supplements/probiotics to assess if ingestion of supplements/probiotics are changing the microbiome/metabolome, and consequently protein production, in a positive manner, for dental care to assess how oral microbiome and metabolome relate to oral health and disease, for post-surgery to determine complications affecting recovery, for soil/water microbiome analysis, for city planning, land development and construction, impact of development on local environmental health, for water microbiome analysis to determine how the local microbial community affects health of fish used as food products, for cosmetics to determine how external application of cosmetics influences overall skin health and skin microbiome assessment to customize skin probiotics that will reduce acne, rosacea, psoriasis, etc.

### Examples

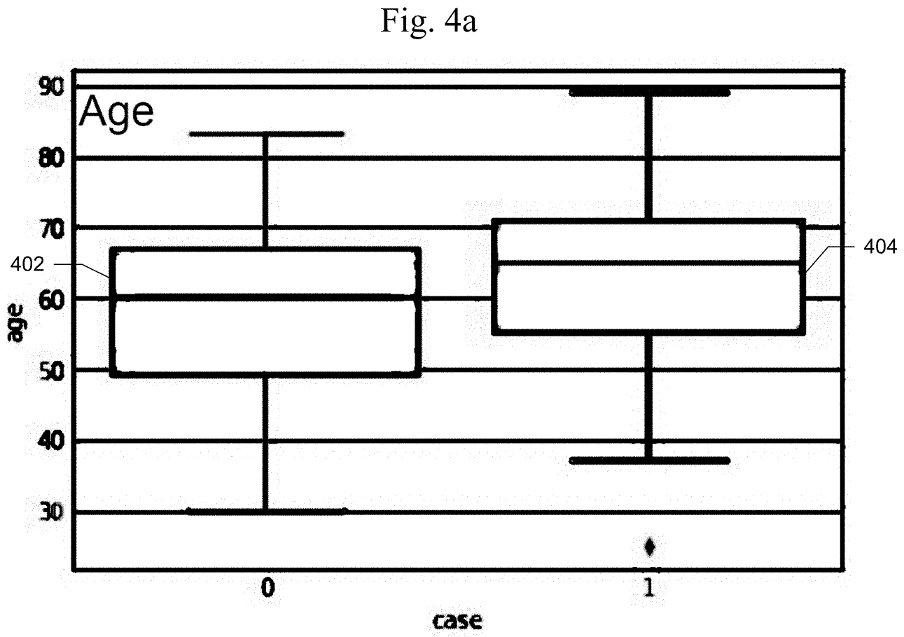

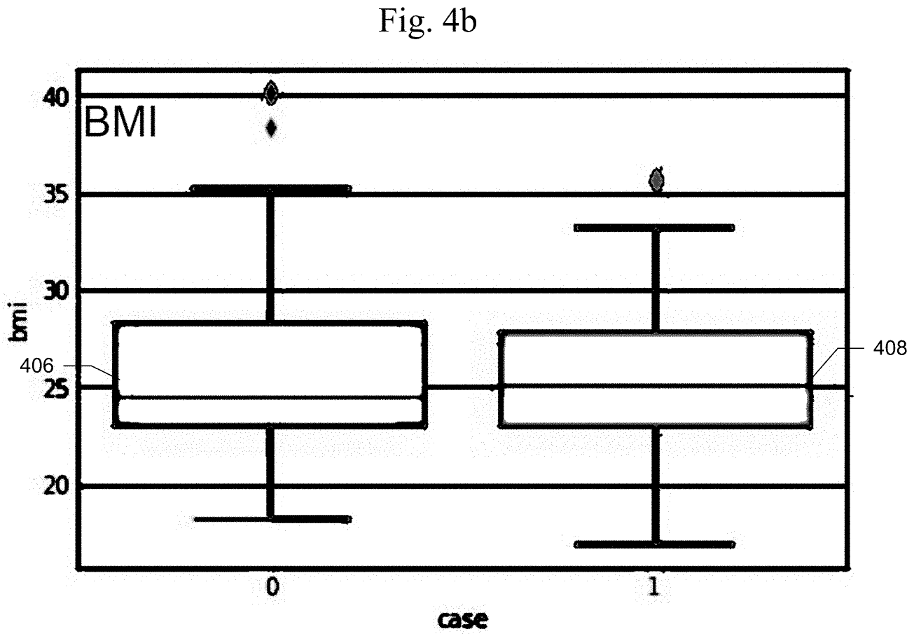

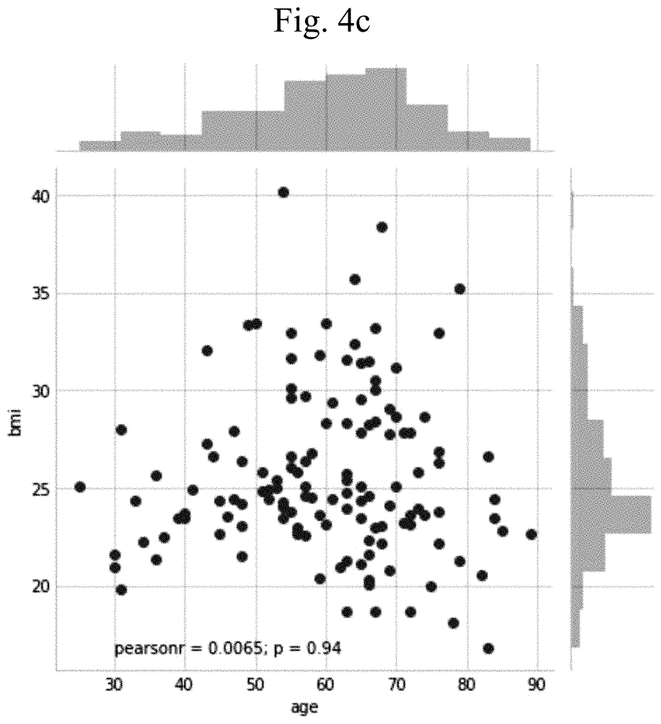

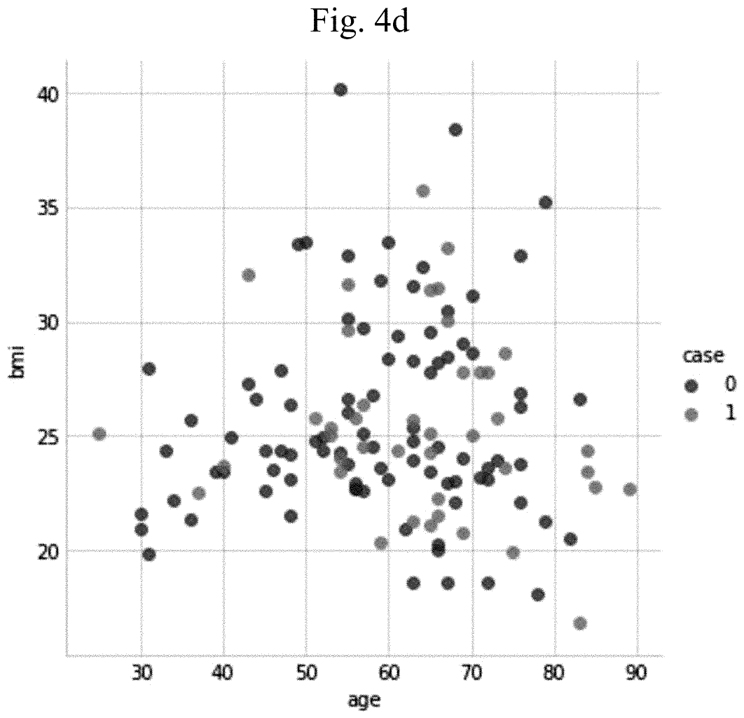

An example of an exploratory analysis of clinical samples for age and BMI matched control and colorectal cancer (CRC) patients is shown in FIGS. 4a, 4b, 4c, and 4d. In the example shown in FIG. 4a, box plots show no significant differences in age of control (0) patients 402 or CRC (1) patients 404. In the example shown in FIG. 4b, box plots show no significant differences in BMI of control (0) patients 406 or CRC (1) patients 408. In FIG. 4c, a scatter plot and histogram show no significant correlation of age and BMI across all control or CRC patients, grouped together. In FIG. 4c, a scatter plot shows no significant correlation of diagnosis of CRC with age or BMI. In this example, each clinical feature is well controlled for in this dataset. There are no significant differences in age, BMI, or sex between control and CRC groups.

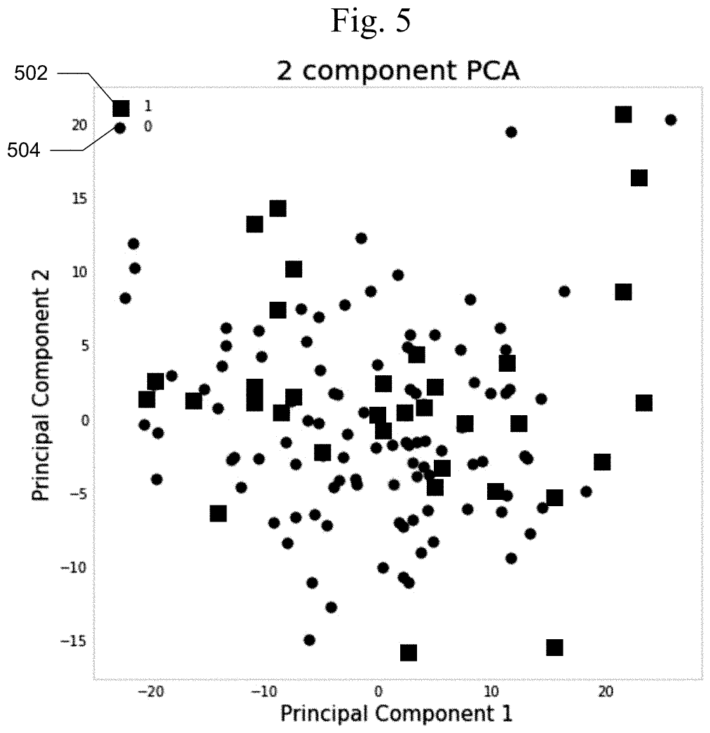

An example of principal component analysis (PCA) is shown in FIG. 5. In this example, all patient samples were analyzed using PCA analysis of all 754 microbial or metabolite features. In this example, the PCA shows the majority variance is across all patient samples with no clear separation or clustering of control (0) 502 or CRC (1) 504 patients. All exploratory analyses indicate that samples are well controlled for confounding clinical characteristics and no obvious differences can be distinguished between control and CRC patients by clinical metadata or features captured.

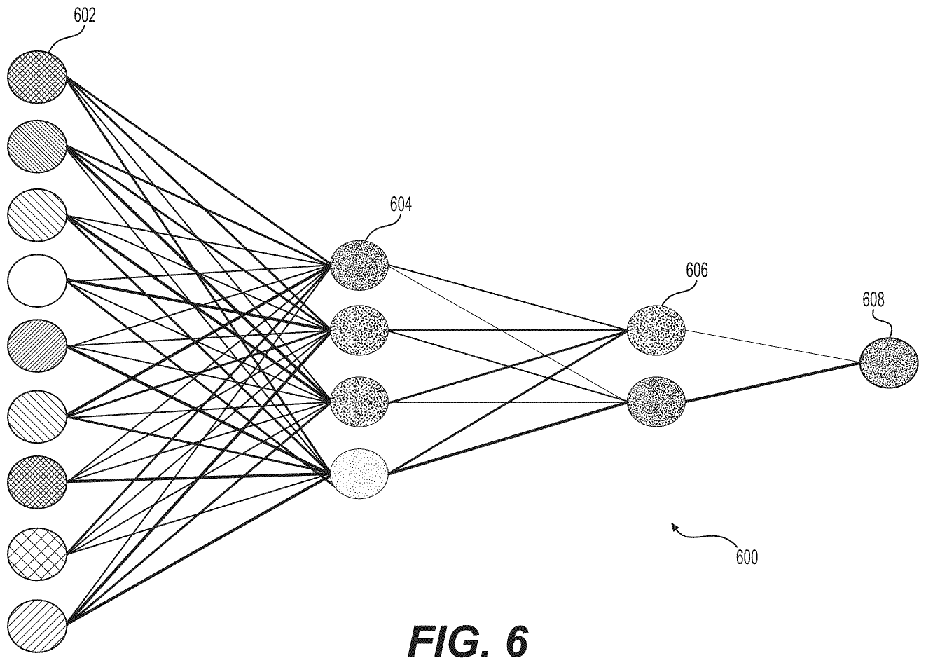

An example of a deep neural network 600, such as may be used to implement 318 in FIG. 3, is shown in FIG. 6. In this example, a dense multilayer perceptron (MLP) was generated for binary classification of the control or CRC patients. A graphical representation of the deep artificial neural network used for modeling data and classification is shown in FIG. 6. Tables 2-5 show a summary of each layer of the deep artificial neural network and relevant hyperparameters used for our MLP. For example, model hyperparameters may include a loss function, such as Binary Cross-Entropy, an optimizer, such as Adaptive Moment Estimation (Adam), a number of iteration epochs, such as 25, and a batch size, such as 32. All 754 microbial, metabolite, and clinical features may be used as input for the binary classification of control or CRC patients in the single output node, which may result in 80601 trainable parameters.

An exemplary configuration of a first layer 602 of deep artificial neural network 600 is shown in Table 2:

TABLE 2
Exemplary first layer of deep artificial network.

| Layer Type | Input |
|---|---|
| # Neurons | 754 |
| Regularization | Dropout 0.2 |
| Activation Function | NA |
| # Parameters | 0 |

An exemplary configuration of a first layer 604 of deep artificial neural network 600 is shown in Table 3:

TABLE 3
Exemplary first layer of deep artificial neural network.

| Layer Type | Dense Hidden |
|---|---|
| # Neurons | 100 |
| Regularization | Dropout 0.2 |
| Activation Function | ReLU |
| # Parameters | 75500 |

An exemplary configuration of a first layer 606 of deep artificial neural network 600 is shown in Table 4:

TABLE 4
Exemplary first layer of deep artificial neural network.

| Layer Type | Dense Hidden |
|---|---|
| # Neurons | 50 |
| Regularization | Dropout 0.2 |
| Activation Function | ReLU |
| # Parameters | 5050 |

An exemplary configuration of a first layer 608 of deep artificial neural network 600 is shown in Table 5:

TABLE 5
Exemplary first layer of deep artificial neural network.

| Layer Type | Output |
|---|---|
| # Neurons | 1 |
| Regularization | NA |
| Activation Function | Sigmoid |
| # Parameters | 51 |

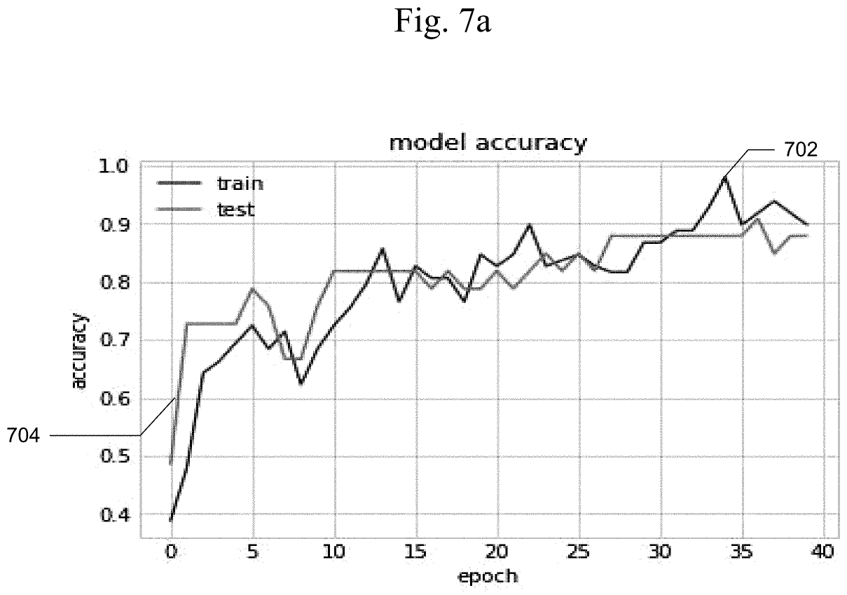

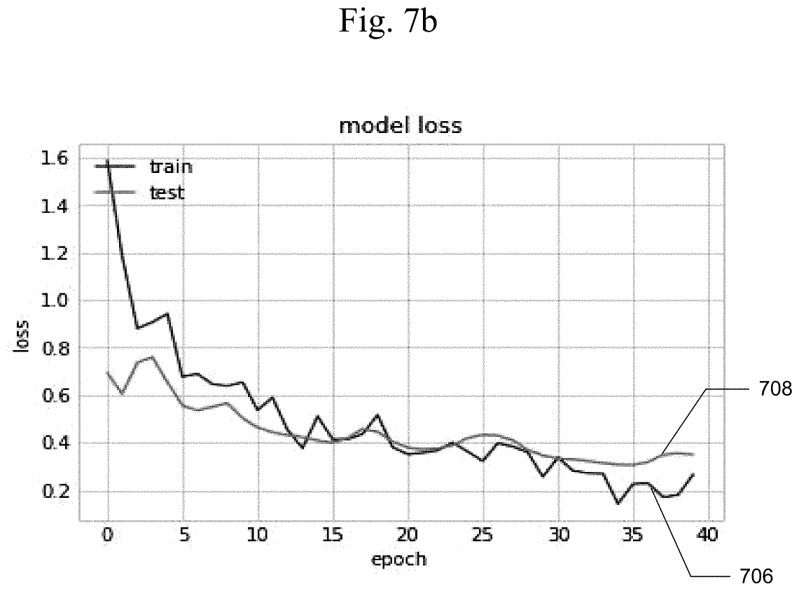

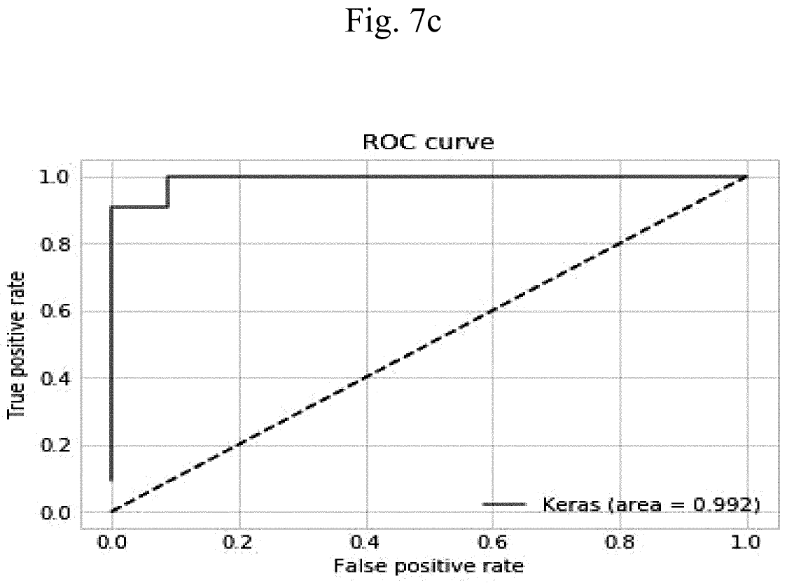

An example of a test of model accuracy is shown in FIG. 7a. In this example, the data was split into training and test sets, training the model on 75% of the data, leaving 25% of the data to test the trained model. The model accuracy and loss were evaluated using these split data over 40 epochs. FIG. 7a shows accuracy of the model, in this example, on training 702 and test 704 sets after the 40 epochs reaches a maximal accuracy, before training and test prediction accuracy divergence, at around 25 epochs. FIG. 7b shows how, in this example, model loss diverges between training 706 and test 708 sets at around 25 epochs. FIG. 7c shows results, for this example, of k-Fold cross validation used to further evaluate the model, setting the number of epoch at 25 based on results from FIGS. 4a and 4b. In this example, a baseline mean accuracy of 78% was achieved using 3-fold cross validation. ROC curves were then generated from this trained model and the predictions on the test data set. A perfectly trained model should have the largest possible area (of 1) under the ROC curve. In this example, the model shows an area of 0.992 (FIG. 7c), which demonstrates that the model can accurately predict the test data from the trained model to a high degree of accuracy.

Identification of important features. From the model, relative feature importance may be determine using, for example, a connection weights process. For example, the top 100 ranked microbial and metabolite features may be extracted for further modeling. Table 6 shows 10 unique microbes and metabolites associated with CRC that were identified by original researchers using multivariate logistic regression.

TABLE 6
Microbes and metabolites associate with CRC.

| Unique microbes | Unique metabolites |
|---|---|
| Fusobacterium | Palmitoyl sphingomyelin |
| Porphyromonas | p Hydroxybenzaldehyde |
| Clostridia | Conjugated linoleic acid (CLA) |
| Lachnospiraceae | p Aminobenzoate (PABA) |
| | Alpha tocopherol |
| | Mandelate |

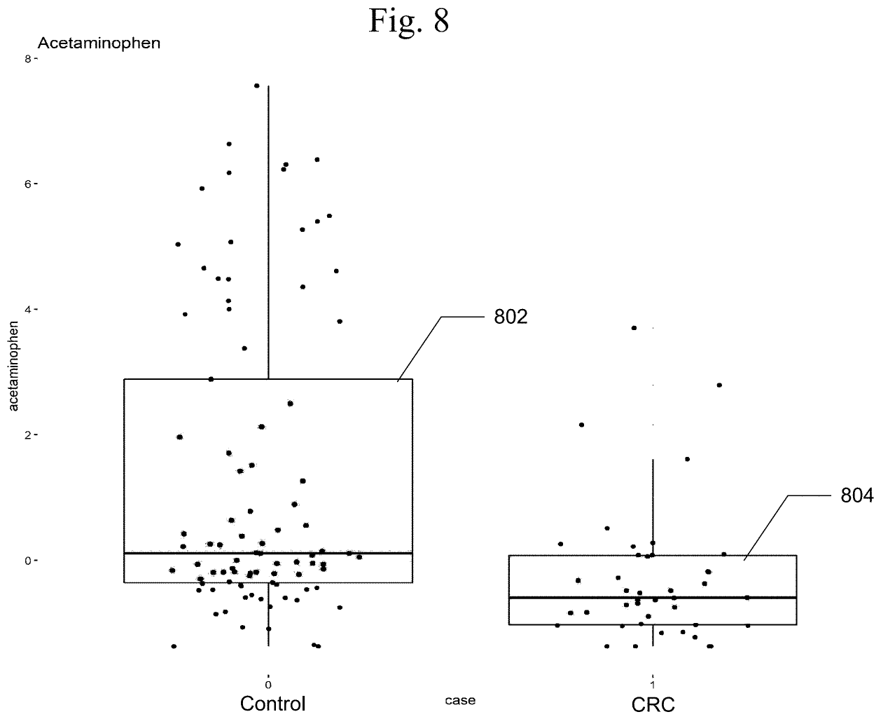

For example, of these ten, nine may be identified in the top 100 microbes and metabolites ranked by the model 802, shown in FIG. 8. In the model, PABA may be the highest ranked overlapping feature by relative importance, only second to ACETAMIDOPHENOL, also known as acetaminophen. ACETAMIDOPHENOL (aka acetaminophen) was also identified in a previous study of the fecal metabolome in the same sample population, as shown FIG. 8. The ability for our model to identify the same unique features associated with CRC as the previous researchers validates our methodology. To determine the biological interpretation and functional importance of these microbes and metabolites and others identified in our model, a network-based methodology may be applied on the top 100 features.

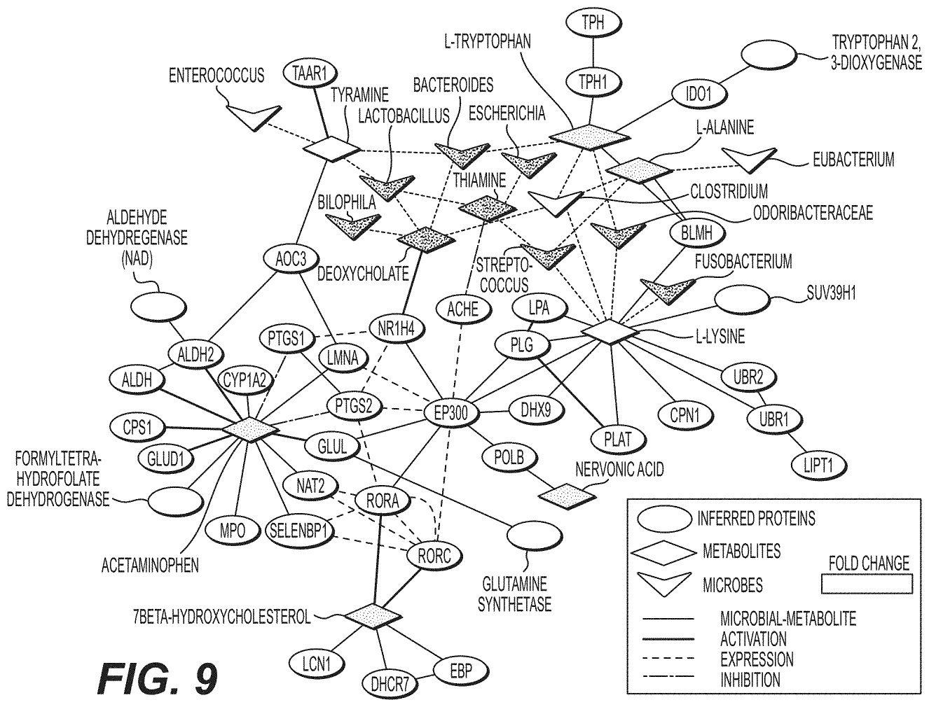

An example of network-based integration of microbiome and metabolome data, and inference of novel proteins is shown in FIG. 9. First, the top 100 metabolites that were identified by the model were used for integration with a protein-protein-metabolite interactome (PPMI). Metabolites were used as the initial network nodes and a single node outgrowth by one edge was used to connect/infer proteins that interact with our metabolites. Once all the proteins that had direct connections to the metabolites were identified, each of the proteins were then further connected in the network graph to each other based on the same PPMI. After removing all self-connecting and duplicate edges, a large subnetwork stood out from the data, which included the top ranked metabolite, by relative importance measure, acetaminophen. This subnetwork was then utilized to connect the top 100 microbial features using a microbe-metabolite interactome (MiMel). FIG. 9 shows an example of a resulting network graph from this analysis. Nodes are colored by the level fold change difference between CRC vs control and edges type and color indicate specific interactions between nodes.

From this network, biological functions may begin to be uncovered, along with how the host-microbiome interactions may influence disease state. Pathways, communities, and hub nodes may be identified to determine the causal effect of having altered levels of microbes or their metabolic products. Additionally, data driven approaches such as Bayesian network analysis will be used to generate directed acyclic graphs from the microbe, metabolite, protein, and clinical/lifestyle data to predict causal inference.

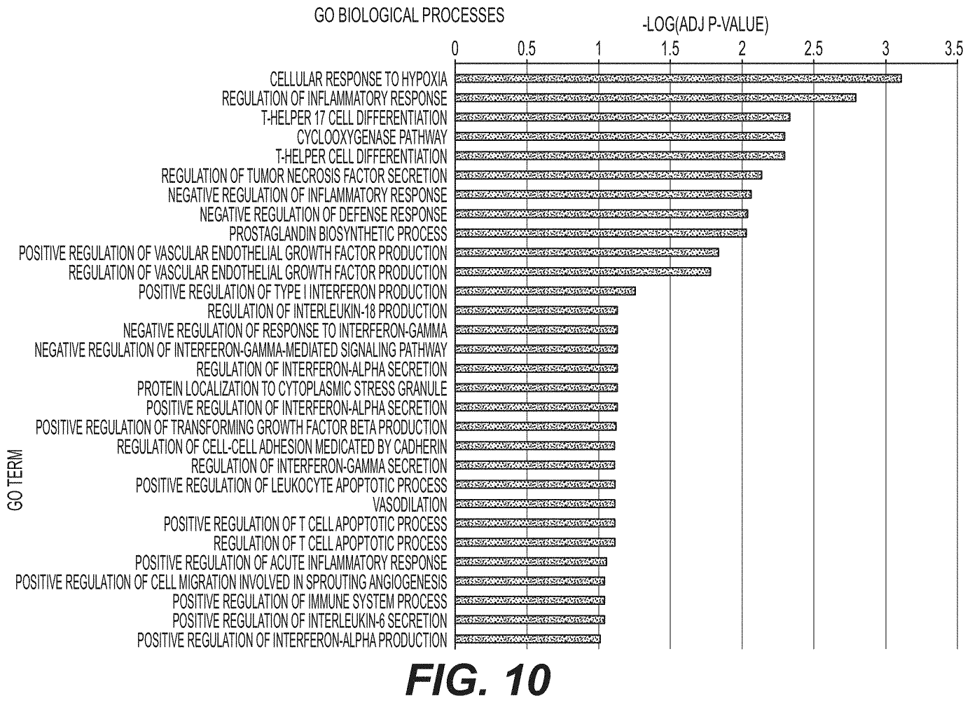

An example of GO enrichment analysis of all inferred proteins from the network analysis shown in FIG. 9 is shown in FIG. 10. The proteins in the network were enriched for GO terms that mainly focused on Immune/inflammation, Angiogenesis, and EMT. FIG. 10 shows the list of enriched GO terms, from the inferred proteins from the network, ranked by −log(adj. p-value), minus terms directly related to metabolism of specific metabolites found in the network. Finding enrichment of processes further validates the model because it usually requires lists of >200 proteins/genes to start to see significant enrichments, so this truly indicates the power of this approach and the significance of each of the proteins in this small network with these identified biological processes. Furthermore, these terms represent the microbial-host interactions that are specifically associated with colorectal cancer. The biological enrichment of immune/inflammation-, angiogenesis-, and EMT-related terms indicates that not only could the gut microbiome be having an impact on the disease, but that it might also be playing a significant role in the actual pathogenesis of the disease and downstream clinical outcomes. These terms further validate the approach and demonstrate that data from the model could be used to identify relevant physiological states, such as CRC, within users and be used to investigate potential therapeutic targets for clinical intervention.

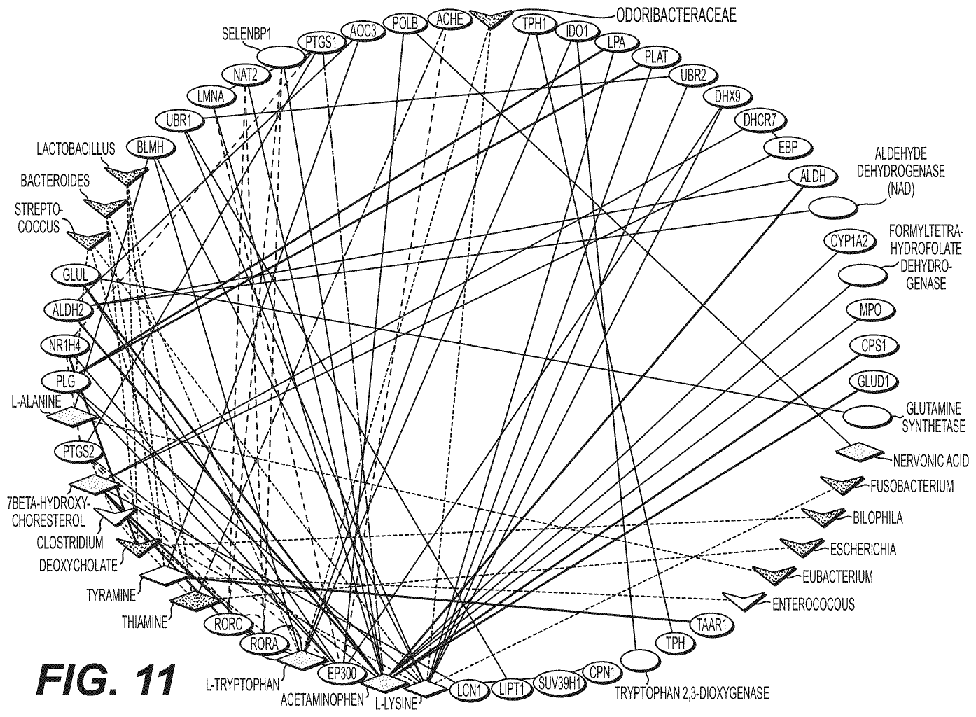

An example of identification of highly influential hub nodes is shown in FIG. 11. To identify important nodes/features in this network, the degree of centrality and betweenness centrality may be calculated. These metrics may determine the degree of connectedness for all nodes and may identify nodes that may act as bridges between subnetwork communities within the larger network. Tables 7 and 8 show the top 5 nodes for each metric. These data taken together with the edge interaction types displayed in the circos plot in FIG. 11 shows that RORA and EP300 could have a significant influence on the expression of many of the other node members of the network. The decrease in acetaminophen levels, indicated by the blue node color, could directly lead to activation and inhibition of many of the other nodes. Of all the microbial features, microbes under the genus Clostridium, which are at higher levels in CRC patients, seem to be playing an important overall role in this network. From these data a novel feature influence scoring algorithm may be utilized to rank order the influence each of these top features has on the overall network biology and combined with the causal inference data at 325 in FIG. 3.

TABLE 7
Top 5 nodes using degree of centrality

| Node | Degree Centrality |
|---|---|
| L-lysine | 0.24137931 |
| acetaminophen | 0.224137931 |
| EP300 | 0.189655172 |
| L-tryptophan | 0.137931034 |
| RORA | 0.103448276 |

TABLE 8
Top 5 nodes using betweenness centrality

| Node | Betweenness Centrality |
|---|---|
| EP300 | 0.456860171 |
| L-lysine | 0.404918383 |
| acetaminophen | 0.247528784 |
| L-tryptophan | 0.15801754 |
| Clostridium | 0.12297336 |

Three of the proteins identified with the model, EP300, RORA, and RORC, have known roles in cancer and metastasis and 2 have cancer therapeutics being developed around their mechanism of action. This validates the ability of the model in identifying proteins with roles in a physiological state, in this case CRC, simply by examining microbial and metabolomic user profiles.

EP300, also known as p300, is an epigenetic molecule that regulates gene expression. Specifically, p300 is critical in regulating cell growth and division and has been shown to prevent continued division of tumorigenic cells. p300 has been identified as having a role in several cancers, including CRC. RORA and RORC are retinoic acid receptor-related orphan receptors that have gained recent interest as therapeutic targets in cancer. Agonists against these proteins have been found to stabilize p53 leading to apoptosis, giving these proteins great therapeutic potential.

Identification of proteins with current therapeutic significance in cancer research from a microbial and metabolomic profile of CRC patients validates the model and confirms the utility of this approach for identifying proteins in the same manner that have currently unknown applications to physiological states. While the physiological state used here as a proof-of-concept was cancer, this approach is not limited specifically to disease states and could be used to even identify proteins from microbial and metabolomic profiles pertaining to innocuous features, such as race, ethnicity, and/or diet and lifestyle. However, as demonstrated, the model can be extended for applications accompanying various clinical or disease-related states, such as, but not limited to, obesity, pharmaceutical use, allergies, and any known or unknown disease, such as, but not limited to, diseases associated with neurology, cardiology, pulmonology, etc. Obtaining such findings from a relatively small data set only further proves the model, as the sophistication and power of this model will improve as the sample size is increased by collecting samples directly from additional people.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A computer-implemented method for determining health, lifestyle, or environmental-related features of microbes and metabolites, the method comprising:
obtaining a biological sample from a subject,
performing quantitative and qualitative physical analysis on the biological sample to generate data identifying species of microbes and metabolites in the biological sample;
annotating and quantifying the data identifying species of microbes and metabolites;
extracting features from the data identifying species of microbes and metabolites;
determining a relative importance of the extracted features using a deep neural network;
generating, using the extracted features and the relative importance of the extracted features, a subnetwork of proteins, metabolites, and microbes by searching a protein-protein metabolite interactome and a microbe-metabolite interactome or using a data driven causal network approach to determine proteins that could be altered in the subject the sample was procured from;
imputing clinical relevance to proteins, metabolites, and microbes present or interacting with the metabolite and microbe samples;
determining a degree of centrality and a degree of betweenness of the imputed proteins, metabolites, and microbes; and
determining a health related influence of each of at least some features.

2. The method of claim 1, wherein the biological samples are selected from the group consisting of fecal samples, skin samples, tissue biopsies, urine, saliva, sputum, mucus, cerebrospinal fluid, and biofilm.

3. The method of claim 1, wherein the performing quantitative and qualitative physical analysis on the biological sample comprises 16s rRNA sequencing or LC/MS.

4. The method of claim 1, further comprising obtaining clinical and lifestyle information from the subject.

5. The method of claim 4, wherein the clinical and lifestyle information is selected from the group comprising age, sex, ethnicity, disease status, weight, diet, drug use, or a combination thereof.

6. A method for determining health-related features of microbes and metabolites, comprising:
obtaining a biological sample from a subject,
identifying and quantifying the species of microbes and metabolites in the biological sample,
ranking the microbes and metabolites based on relative importance, and
determining interactions between ranked microbes and metabolites and proteins to identify proteins involved in a health, lifestyle, or environmental-related condition.

7. The method of claim 6, wherein ranking the microbes and metabolites comprises using a deep neural network.

8. The method of claim 6 wherein determining interactions between ranked microbes and metabolites and proteins comprises using a protein-protein metabolite interactome and a microbe-metabolite interactome, and data driven causal connections.

9. The method of claim 6, wherein identifying and quantifying the species of microbes and metabolites in the biological sample comprises 16s rRNA sequencing or LC/MS.

10. The method of claim 6, wherein the biological samples are selected from the group consisting of soil samples, fecal samples, skin samples, tissue biopsies, urine, saliva, sputum, mucus, cerebrospinal fluid, and biofilm.

11. A system comprising a processor, memory accessible by the processor, and computer program instructions stored in the memory and executable by the processor to perform:
receiving data identifying species of microbes and metabolites in a biological sample, the data generated by: obtaining a biological sample from a subject and performing quantitative and qualitative physical analysis on the biological sample to generate data;
annotating and quantifying the data identifying species of microbes and metabolites;
extracting features from the data identifying species of microbes and metabolites;
determining a relative importance of the extracted features using a deep neural network;
generating, using the extracted features and the relative importance of the extracted features, a subnetwork of proteins, metabolites, and microbes by searching a protein-protein metabolite interactome and a microbe-metabolite interactome or using a data driven causal network approach to determine proteins that could be altered in the subject the sample was procured from;
imputing clinical relevance to proteins, metabolites, and microbes present or interacting with the metabolite and microbe samples;
determining a degree of centrality and a degree of betweenness of the imputed proteins, metabolites, and microbes; and
determining a health related influence of each of at least some features.

12. The system of claim 11, wherein the biological samples are selected from the group consisting of fecal samples, skin samples, tissue biopsies, urine, saliva, sputum, mucus, cerebrospinal fluid, and biofilm.

13. The system of claim 11, wherein the performing quantitative and qualitative physical analysis on the biological sample comprises 16s rRNA sequencing or LC/MS.

14. The system of claim 11, further comprising obtaining clinical and lifestyle information from the subject.

15. The system of claim 14, wherein the clinical and lifestyle information is selected from the group comprising age, sex, ethnicity, disease status, weight, diet, drug use, or a combination thereof.
